(19) United States
(12) Patent Application Publication
(10) Pub. No.: US 2003/0064521 A1
(43) Pub. Date: Apr. 3, 2003

(54) Method for ending point detection during etching process

(76) Inventors: Zhijian Lu, Poughkeepsie, NY (US); Stephen H. Rahn, Hoehenkirchen-Slegertsbrunn (DE)

Correspondence Address: Infineon Technologies North America Corp., c/o Siemens Corporation, Intellectual Property Department, 186 Wood Avenue South, Iselin, NJ, 08830, US

(21) Appl. No.: 09/966,923

(22) Filed: Sep. 28, 2001

(51) Int. Cl. G01N 33/00

(52) U.S. Cl. 436/55; 436/73; 436/173

(57) ABSTRACT

A method for detecting an ending point during an etching process in semiconductor fabrication. In general, an implantation technique in combination with chemical analysis of the implants is used for ending point detection. In one aspect, a method for detecting an endpoint of an etch process comprises the steps of implanting a dopant into a material at a reference depth, detecting a concentration of the dopant in an etching environment as the material is etched, and determining that the material has been etched to the reference depth when peak concentration of the dopant is detected.

### BACKGROUND

1. Technical Field

The present invention relates generally to a method for manufacturing semiconductor devices and, in particular, to an improved ending point detection method using dopant implantation and chemical analysis of the concentration of the implant in an etching environment to determine an endpoint of the etch process.

2. Description of Related Art

Typically, the manufacture of semiconductor devices comprises a sequence of various processing steps including, for example, depositing a layer of material over a substrate, forming an etching mask on the surface of the deposited material, etching the deposited material to define a structure over the substrate, and then removing the etching mask, which sequence may be repeated any number of times during the manufacture of a typical semiconductor device. Techniques for monitoring the progress of these processing steps is an important component in the manufacture of semiconductor devices so as to minimize costs of manufacture and increase yield.

The etching process, which may comprise one of various wet and dry etching methods, is important step in the manufacture of semiconductor devices. Dry etching is a generic term that encompasses etching techniques in which gases, as opposed to liquid chemicals, are the primary etch medium. Examples of dry etching techniques used in the manufacture of solid state electronic devices are ion beam milling, plasma etching, and reactive ion etching (“RIE”).

The use of process monitoring techniques to control etching processes are important because of the critical nature of the etching process. A typical etching process is performed to remove the portion of a layer of deposited material that is exposed through the openings in an etching mask. The point at which the etching process is complete is referred to as the “endpoint” for the etching process. If the etching process proceeds for too long, over etching occurs which may damage, e.g., an integrated circuit. However, prematurely terminating the etching process can result in an incomplete etch and consequently, the improper formation of circuit components. As the size or critical dimension of integrated circuits is reduced below the sub micron level, the proper determination of the endpoint of the etching process becomes more and more difficult. Indeed, endpoint detection techniques have become increasingly sophisticated as design rules shrink and greater control over etch parameters becomes necessary.

There are various methods that may be used for determining an endpoint for an etch process. One method comprises determining the average etch rate of a dry etch process, and then estimating the etch time needed to remove the desired amount of material based on the etch rate. One disadvantage associated with this method is that there is no way to compensate for run-to-run fluctuations in etch rate. The etch rate may vary between runs because of variations in material properties, film thickness, or processing conditions. Consequently, this approach of using a predetermined length of time to control the etching process is not very reliable, especially in the case of wafers designed with small critical dimensions.

Typically, optical-based ending point systems, which use optical emission detectors and optical signal analysis and methods, are used in conjunction with dry etching techniques for the manufacture of semiconductor devices. For instance, optical end point detection techniques such as ellipsometry and laser reflectometry measure layer thicknesses directly and provide real-time localized endpoint determinations. These methods involve determining when the endpoint of the process has been reached by measuring the thickness of the film being etched. Unfortunately, these optical methods may be adversely affected by the surface morphology of the layer being etched. Furthermore, the sensitivity of these optical methods decreases when the layer being etched is on the order of a few angstroms thick, because the optical interference effects for such thin layers are quite small. Thus, these optical schemes produce some degree of over etching.

Optical emission spectroscopy is another widely used optical-based endpoint detection method because it is easy to implement. This method is used for detecting the presence of gaseous byproducts of a plasma etching processes within an etching chamber, for example. During plasma etching, an etchant gas is released into a plasma processing chamber. During the etching process, etch species and reactants in the processing chamber emit light when their excited electrons change energy states. Each species produces a unique wavelength of light, and the intensity of each wavelength of light emitted from the plasma is related to the concentration of that species within the plasma. As a wafer is being etched, a reaction equilibrium is generally sustained within the plasma until the layer that is being etched starts to clear or be fully removed. At this point, the increase in the concentration of the etchant species and the decrease in the concentration of the reaction product species causes the light intensities associated with these species to increase or decrease. By measuring the light emission intensity change associated with the chemical species in the plasma, an endpoint for the etching process can be determined.

The reactant chamber typically comprises a window that allows the light produced by the reactions within the chamber to be detected by photo-diode optical sensors outside of the chamber. The optical sensor produces an electrical signal that represents the intensity of light. Optical signal analysis and detecting algorithm are then used to determine the endpoint for the etching process. Control signals are then generated to stop the etching process. In one method, the sensor is used to detect the intensity of a certain wavelength of light that is produced by one of the reactants of the etching process. Generally, when the intensity of the wavelength of light crosses a predetermined threshold, the computer signals that the endpoint has been reached. In another method, the shape of a curve representing the changes in the intensity of a particular wavelength of light that is produced by one of the reactants is used to determine the endpoint. In this method, the computer monitors the electrical signal provided by the optical sensor and compares the shape of the signal over time to a predetermined shape. Once a match is found, the computer signals the endpoint for the etching process.

There are disadvantages associated with Optical emission detection methods. For example, the window in the reactant chamber can become cloudy due to deposits of polymers or other reaction products within the plasma on the inner surface of the window, which can reduce the intensity of the light emitted from the chamber causing inaccurate detection of the endpoint. Another disadvantage is that, as the critical dimensions of the devices being produced on the wafer become smaller and smaller, the optical signals or light produced by the reactants becomes weaker and weaker. This makes it more and more difficult to discriminate between the background noise (e.g. light from other sources) and the light produced by the reactant that is being used to detect the endpoint. This may result in not detecting the proper endpoint and may significantly reduce the yield for a given etching process. Because of the very high cost of producing wafers with complex integrated circuits, this reduced yield can be very costly.

Improved endpoint detection techniques are continually developed, which provide sensitive and direct thickness measurements, and which are compatible with dry etch processes and can be implemented as in-situ monitoring tools. In view of the foregoing disadvantages associated with conventional end point detection schemes, an improved ending point detection method is highly desirable.

### SUMMARY OF THE INVENTION

The present invention is directed to an improved method for detecting an ending point during an etching process in semiconductor fabrication. In general, an implantation technique in combination with chemical analysis of the implants is used for ending point detection.

In one aspect of the present invention, a method for detecting an endpoint of an etch process comprises the steps of:

implanting a dopant within a semiconductor film at a desired implant depth and concentration; and

chemically analyzing a concentration of the implanted dopant released from the semiconductor film during an etch process to determine an endpoint for the etch process.

In another aspect, a method for detecting an endpoint of an etch process comprises the steps of:

implanting a dopant into a material at a reference depth;

detecting a concentration of the dopant in an etching environment as the material is etched; and

determining that the material has been etched to the reference depth when peak concentration of the dopant is detected.

In another aspect, the reference depth is either approximately the same as a desired etch distance or is less than a desired etch distance.

In yet another aspect, the step of detecting comprises detecting the concentration of compound formed from the dopant during the etching process.

These and other objects, features and advantages of the present invention will be described or become apparent from the following detailed description of p referred embodiments, which is to be read in connection with the accompanying drawings.

### BRIEF DESCRIPTION OF DRAWINGS

FIG. 1 is a cross-sectional view of a portion of an exemplary semiconductor device;

FIG. 2 is a diagram illustrating one step of an ending point detection process according to the present invention, whereby a dopant is implanted into material A of the semiconductor device of FIG. 1;

FIG. 3 is a diagram illustrating an exemplary concentration of an implanted dopant as a function of the thickness of material A and reference depth; and

FIG. 4 is a diagram illustrating other steps of an ending point detection process according to the present invention, wherein the dopant concentration is detected during an etching process to determine when the etching process has reached a reference depth.

### DETAILED DESCRIPTION OF PREFERRED EMBODIMENTS

The present invention is directed to an improved method for detecting an ending point during an etching process in semiconductor fabrication. In general, an implantation technique in combination with chemical analysis of the implants is used for ending point detection. A preferred method described below involves implanting a dopant in a layer that is to be etched, wherein the region below the surface of the layer having the greatest concentration of the dopant provides a reference depth for the etch process. During etching, a chemical analysis is performed to detect the concentration of the dopant or the concentration of another compound that is formed from the implanted dopant during the etching process. An ending point detection process is performed by determining when the concentration of detected dopant (or other compound) corresponds to the known concentration of dopant near the reference depth. An ending point detection process as described herein may be used in conjunction with any suitable wet-etching or dry-etching technique such as in a RIE (reactive ion etching) process.

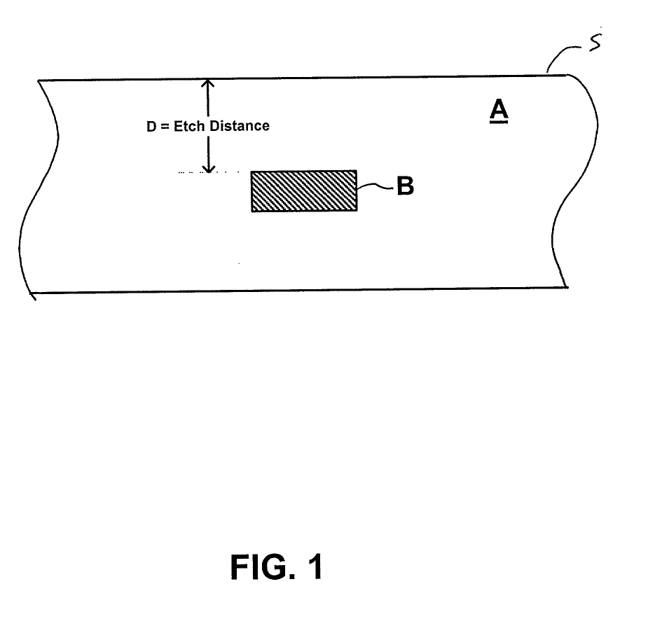

FIG. 1 illustrates a cross-sectional view of a portion of an exemplary semiconductor device having a first layer (comprising material A) and a second layer (comprising material B) embedded within the first layer at a distance D below the surface of the first layer. In the exemplary embodiment described herein, it is assumed that the first layer is to be etched down to the upper surface of the second layer (i.e., the distance D represents the etching distance). It is to be understood that the structure shown in FIG. 1 is for illustrative purposes only, and that an ending point detection method according to this invention may be applied for various architectures.

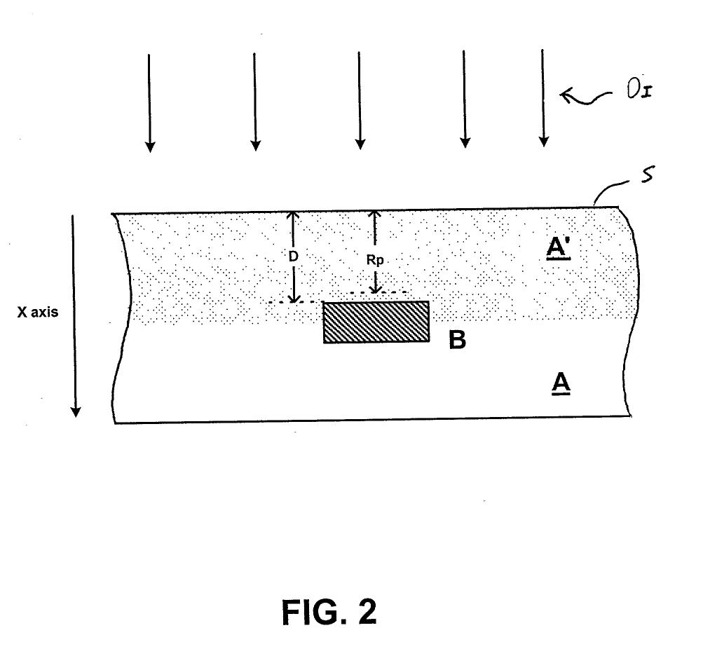

Referring now to FIG. 2, a diagram illustrates one step in an ending point detection method according to the present invention wherein a dopant D_{I} (ions, atoms, etc.) is first implanted into the layer to be etched (material A) using any suitable conventional implantation process. More specifically, a dopant D_{I} (comprising, N, H, B, P, or Aₛ for example) is implanted in material A with an implant depth (or projected range), denoted as Rₚ, wherein Rₚ is equal to or smaller than the distance from the top surface S of the first layer A to the upper surface of the second layer B (i.e., the etching distance D) under a given ion energy and dose. In FIG. 2, the region A′ denotes the portion of region A comprising implanted dopant.

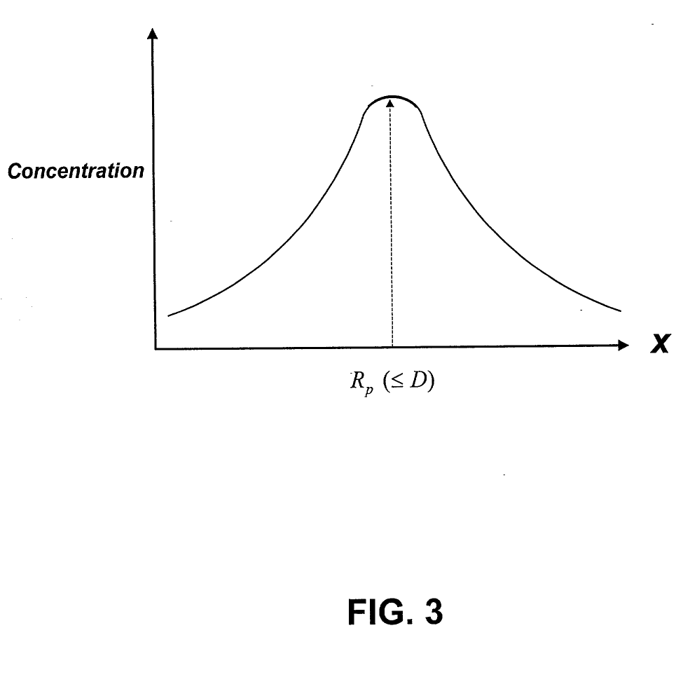

In a preferred embodiment, the peak concentration of the dopant is located at the depth Rₚ, which, in a preferred embodiment, is less than or equal to the etch distance D. FIG. 3 is a graphical diagram that illustrates the dopant concentration profile (y axis) as a function of the thickness of the first layer A (x axis). As shown, the peak concentration of the dopant is located at the depth Rₚ below the surface S (FIG. 2), which provides a reference depth for the etching process. Indeed, since the implantation depth is well controlled, the peak concentration of the dopant within the material to be etched provides an exact reference depth for ending point detection during the etch process. As explained below, as the first layer A is etched, the change in the dopant concentration in the etch environment (e.g., plasma) is detected. When the anticipated peak concentration of dopant is detected, it is known that the reference depth Rₚ has been reached, which is baseline for further etching.

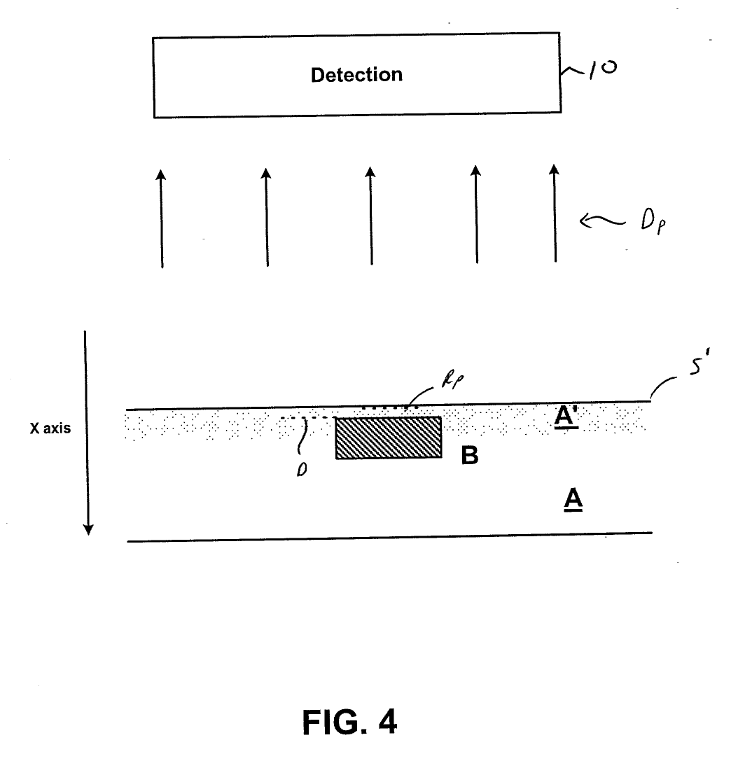

More specifically, referring to FIG. 4, a diagram illustrates additional steps comprising an ending point detection process according to the invention. During an etching process (e.g., reactive ion etching), the first layer A will be etched such that the original surface S (shown in FIG. 2) will be etched at a certain etch rate. During etching, the concentration of the dopant species (or compounds comprising the dopant), denoted as Dₚ, will be detected based on, e.g., the concentration profile as a function of material thickness as shown in FIG. 3. As the etched surface S′ approaches and meets the reference depth Rₚ, the detection process 10 will detect the peak concentration of the dopant species, thereby indicating that the etch process has reached the reference depth Rₚ. In one embodiment, mass spectrometry is used for real-time detection of the concentration of a target chemical species in the etching chamber.

The reference depth Rₚ essentially provides a reference point for the etching process. For instance, if the reference depth Rₚ was selected to be equal (or approximately equal) to the etch distance D, then no further etching will be performed. If the reference depth Rₚ was selected to be less than the etch distance D, then based on, e.g., the known etch rate and the distance between the reference depth Rₚ and etch distance D, further etching may be performed to reach the desired etch distance D. Thus, by detecting the dopant concentration change, particularly the peak concentration corresponding to the reference depth Rₚ, the etching process can be end-pointed.

Those of ordinary skill in the art will readily understand that the type of dopant that is implanted will vary based on factors such as the type of etch process (e.g., plasma etch) or the type of detector and/or detection process. Further, the dopant type may vary based on the etch chemistry of the material to be etched (i.e., a certain dopant species is preferably not implanted if the material to be etched comprises such species). Furthermore, detection may involve detecting the concentration of either the implanted species or a product formed during the etch process that comprises the implanted species.

By way of example, assume the layer to be etched comprises a polysilicon film that is doped with hydrogen at a given implantation reference depth Rₚ. Assume further that a chlorine gas is used to etch the polysilicon in a dry etching process. During an dry-etch process, as the polysilicon layer is etch, the hydrogen will combine with the chlorine gas to form HCl (hyrdrogen chloride). The concentration of HCl in the plasma can be detected using a mass spectrometer as is well known in the art. As the etching approaches the peak concentration of hydrogen at the reference depth Rₚ within the polysilicon layer, the concentration of HCl in the plasma will be detected at the peak concentration, thereby indicating that the reference depth has been reached.

In other embodiments, the type of implanted species and detected species will be specific to the type of etch process and the dopant used. For instance, the implant may be P and the etch process may generate a product such as PH₃, wherein the peak concentration of PH₃ is detected. Further, where hydrogen is the implanted species, depending on the etch process, H₂O or HBR concentration may be detected. Based on the teachings herein, those of ordinary skill in the art can readily envision various applications of the present invention and its application with various etching processes.

Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention. All such changes and modifications are intended to be included within the scope of the invention as defined by the appended claims.

What is claimed is:

1. A method for detecting an endpoint of an etch process, comprising the steps of:
implanting a dopant within a semiconductor film at a desired implant depth and concentration; and
chemically analyzing a concentration of the implanted dopant released from the semiconductor film during an etch process to determine an endpoint for the etch process.

2. the method of claim 1, wherein the implant depth is approximately equal to an etch distance.

3. The method of claim 1, wherein the endpoint of the etch process is determined based on a peak concentration of the implant dopant in an etch plasma.

4. A method for detecting an endpoint of an etch process, comprising the steps of:
implanting a dopant into a material at a reference depth;
detecting a concentration of the dopant in an etching environment as the material is etched; and
determining that the material has been etched to the reference depth when peak concentration of the dopant is detected.

5. The method of claim 4, wherein the reference depth is approximately the same as a desired etch distance.

6. The method of claim 4, wherein the reference depth is less than a desired etch distance.

7. The method of claim 4, wherein the step of detecting comprises detecting the concentration of compound formed from the dopant during the etching process.

8. The method of claim 4, wherein the step of detecting comprises mass spectrometry.

9. The method of claim 4, wherein the etching environment comprises a plasma.

10. The method of claim 4, wherein the dopant comprises one of N, H, O, B, P, As, and S.

11. The method of claim 4, wherein the etch process comprises a wet etch.

12. The method of claim 4, wherein the etch process comprises a dry etch.
